(19) United States
(12) Patent Application Publication
(10) Pub. No.: US 2021/0225462 A1
(43) Pub. Date: Jul. 22, 2021

(54) Method Of Synthesizing Chemical Compounds

(71) Applicant: EMD Millipore Corporation, Burlington, MA (US)

(72) Inventors: Karol Molga, Warsaw (PL); Piotr Dittwald, Warsaw (PL); Bartosz A. Grzybowski, Warsaw (PL)

(21) Appl. No.: 17/130,659

(22) Filed: Dec. 22, 2020

Related U.S. Application Data
(60) Provisional application No. 62/961,767, filed on Jan. 16, 2020.

(51) Int. Cl. G16C 20/10

(52) U.S. Cl. CPC G16C 20/10

(57) ABSTRACT

By keeping track of lists of specific bonds that are to be preserved, a computer program is able to design synthetic routes to create a target compound that avoid that previously published or patented approaches. This may allow the exploration of lower cost or more efficient methods of creating known compounds, or may allow the synthesis of new compunds without the use of patented compunds. Examples of computer-designed syntheses relevant to medicinal chemistry are provided in which the machine avoids “strategic” disconnections common to industrial patents and/or is forced to use different starting materials.

This application claims priority of U.S. Provisional Patent Application Ser. No. 62/961,767, filed Jan. 16, 2020, the disclosure of which is incorporated herein by reference in its entirety.

This disclosure describes systems and methods for synthesizing pathways to create chemical compounds, also referred to as retrosynthetic analysis.

### BACKGROUND

Programming a computer to plan multistep chemical syntheses leading to nontrivial targets has been an elusive goal for over five decades. Only recently has the first comprehensive validation of in silico synthetic predictions has been provided. Specifically, one software application, referred to commercially as Synthia™, available from MilliporeSigma, designed, without any human supervision, complete pathways leading to eight structurally diverse and medicinally relevant targets. These theoretical pathways were subsequently executed in the laboratory, offering substantial improvements over previous approaches or providing the first documented routes to a given target.

Knowing that retrosynthesis is achievable, one can consider expanding the scope of automated retrosynthetic design modalities. One of the interesting possibilities is to challenge the software application to search for pathways significantly different than those already published or patented. This may be useful in finding lower cost or more efficient methods of producing known target molecules. Alternatively, it may be used to create new target molecules without relying on the use of patented compounds.

In principle, this can be done by excluding specific intermediates or reaction types along the route. In practice, however, creating lists of “excluded” substances or reaction types is not only cumbersome for the software's user but can also be of limited value. Indeed, this approach does not prevent the software application from using intermediates that are chemically equivalent to those present in original routes or alternative methodologies resulting in identical retrosynthetic disconnections.

Therefore, it would be beneficial if there was a system and method that provided a convenient and robust approach in which lists of bonds specified in the target may be designated as “preserved” bonds, which are propagated along entire computer-designed pathways. In particular, by “preserving” bonds that were essential in previously patented routes, the software application would be forced to design qualitatively different synthetic plans.

### SUMMARY

By keeping track of lists of specific bonds that are to be preserved, a computer program is able to design synthetic routes to create a target compound that avoid previously published or patented approaches. This may allow the exploration of lower cost or more efficient methods of creating known compounds, or may allow the synthesis of new compunds without the use of patented compunds. Examples of computer-designed syntheses relevant to medicinal chemistry are provided in which the machine avoids “strategic” disconnections common to industrial patents and/or is forced to use different starting materials.

According to one embodiment, a method for performing retrosynthesis on a target compound wherein certain bonds are preserved is disclosed. The method comprises identifying bonds in the target compound that are to be preserved; setting the target compound to a retron; performing a first retrosynthesis search on the retron to find a set of synthons; determining if the bonds are preserved across the set of synthons; discarding the set of synthons if the bonds are not preserved; and if the bonds are preserved, setting the set of synthons to the retron and repeating the performing, determining, and discarding steps. In certain embodiments, the certain bonds are preserved so as to avoid a particular synthon. In some further embodiments, the particular synthon is a patented compound. In certain embodiments, the setting, performing, determining and discarding steps are repeated until all synthons meet user specified criteria. In certain further embodiments, the user specified criteria comprises all synthons are commercially available.

According to another embodiment, software program, disposed on a non-transitory storage media is disclosed. The the software program comprising instructions, which when executed by a processing unit perform retrosynthesis on a target compound wherein certain bonds are preserved, by: identifying bonds in the target compound that are to be preserved; setting the target compound to a retron; performing a first retrosynthesis search on the retron to find a set of synthons; determining if the bonds are preserved across the set of synthons; discarding the set of synthons if the bonds are not preserved; and if the bonds are preserved, setting the set of synthons to the retron and repeating the performing, determining, and discarding steps. In certain embodiments, the setting, performing, determining and discarding steps are repeated until all synthons meet user specified criteria. In certain further embodiments, the user specified criteria comprises all synthons are commercially available. In some embodiments, the bonds of each synthon in the set of synthons are identified and wherein determining if the bonds are preserved across the set of synthons comprises comparing the bonds identified in each synthon in the set of synthons to the bonds that are to be preserved. In some embodiments, the bonds that are to be preserved are identified based on labels assigned in a SMILES string.

### BRIEF DESCRIPTION OF THE DRAWINGS

For a better understanding of the present disclosure, reference is made to the accompanying drawings, in which like elements are referenced with like numerals, and in which:

FIG. 1 shows a representative system for performing the retrosynthesis;

FIGS. 2A-2D show various compounds with “preserved bonds”;

FIG. 3 shows the process of entering “preserved bonds” in a target molecule to generate synthesis paths;

FIGS. 4A-4B shows two examples with a repeated intermediate compound;

FIG. 5 shows the pseudocode related to the algorithm used to insure that “preserved bonds” remain intact; and

FIGS. 6A-6E compare conventional syntheses of the antibiotic linezolid with new synthetic routes with new synthetic routes found when preserving specific bonds.

### DETAILED DESCRIPTION

The present disclosure represents an advancement in the retrosynthesis of chemical compounds. As described above, most current approaches result in pathways that are well known and may be already patented or otherwise protected. This disclosure presents a method for creating compounds without the use of patented processes or compounds. Such a method may be beneficial in finding lower cost or more efficient methods of creating a known compound. Alternatively, this method may be beneficial in creating new compounds without the use of patented reactants.

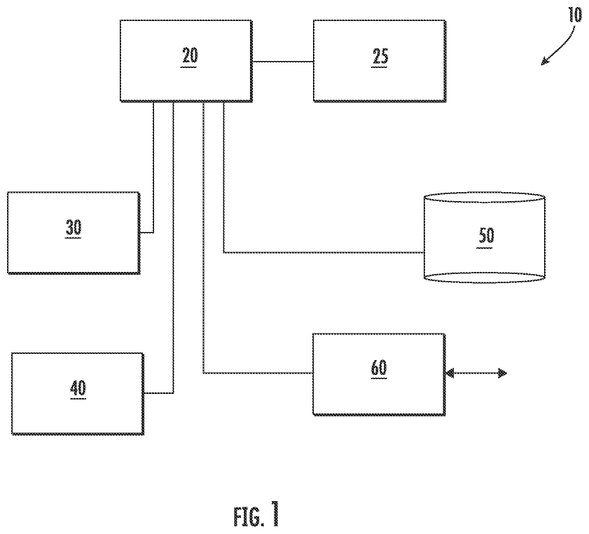

The present disclosure describes a system, method and software application that allow for retrosynthesis analysis that includes constraints to avoid certain compounds or processes. The software application may be written in any suitable language and may be executed on any system. The software application comprises one or more processing blocks. Each of these processing blocks may be a software module or application that is executed on a computer or other processing unit. A representative system 10 that executes the software application is shown in FIG. 1. The processing unit 20 can be implemented in numerous ways, such as with dedicated hardware, or with general purpose hardware, such as personal computers, that is programmed using microcode or software to perform the functions recited herein. A local memory device 25 may contain the software application and instructions, which, when executed by the processing unit, enable the system to perform the functions described herein. This local memory device 25 may be a non-volatile memory, such as a FLASH ROM, an electrically erasable ROM or other suitable devices. In other embodiments, the local memory device 25 may be a volatile memory, such as a RAM or DRAM. The system 10 also comprises a data store 50. The data store 50 may be used to store large amounts of data, such as lists of reaction rules, lists of commercial compounds and their prices per gram. Additionally, the system 10 may include a user input device 30, such as a keyboard, mouse, touch screen or another suitable device. The system may also include a display device 40, such as a computer screen, LED display, touch screen or the like. The data store 50, the user input device 30 and the display device 40 are all in communication with the processing unit 20. In some embodiments, the system 10 may also have a network interface 60, in communication with an external network, such as the internet, which allows the processing unit 20 to access information that is stored remotely from the system.

The data store 50 may store a vast knowledge base of methodologies that describe known reactions, including information about, for example, reaction classes, providing contextual information about potential reactivity conflicts, protection requirements, and others. These “reaction rules” provide the basic moves used by the algorithms to search enormous trees of synthetic possibilities. The synthetic positions on these trees are evaluated by the algorithm according to reaction- and chemical-scoring functions. The exploration of the trees is further guided by multi-step strategies overcoming local complexity barriers as well as a host of quantum-mechanical and molecular-mechanics routines that inspect the structures of the intermediates created during planning. In one embodiment, the data store 50 may include in excess of 60,000 reaction rules. In addition, the system 10 may have access to diverse collections of starting materials. This information may be stored in the data store 50 or may be accessible to the processing unit 20 via the network interface 60. In one embodiment, information regarding more than 7 million literature-known substances is available to the processing unit. This information may also include pricing per gram for at least some of these substances.

In one embodiment, tracking of not-to-be-altered motifs may be implemented by checking if the desired substructure remains intact in the synthons of each reaction that is considered by the software application. A synthon is a destructural unit within a molecule which is related to a possible synthetic operation.

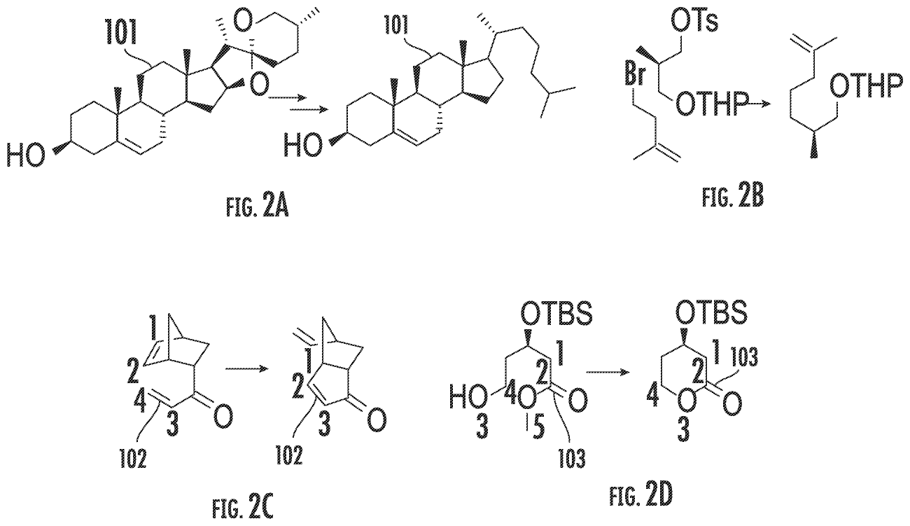

However, this approach works well only when the substructures are larger and unique. FIG. 2A shows a substructure 101 that is readily identified so that it may be preserved. With smaller motifs, one rapidly runs into problems associated with their non-uniqueness. For example, looking at FIG. 2B, it is not readily obvious which of the seven C-C bonds in the main skeleton of the ZK-EPO intermediate must be preserved. In addition, a motif might appear to remain intact but, in reality, changes during the reaction. For example, FIG. 2C shows olefin metathesis where motif 102 appears to remain intact but has been changed. Specifically, the double bond C2=C3 appears to be preserved, but in reality, it has become disconnected. In similar change in motif 103 is shown in the lactonization reaction shown in FIG. 2D.

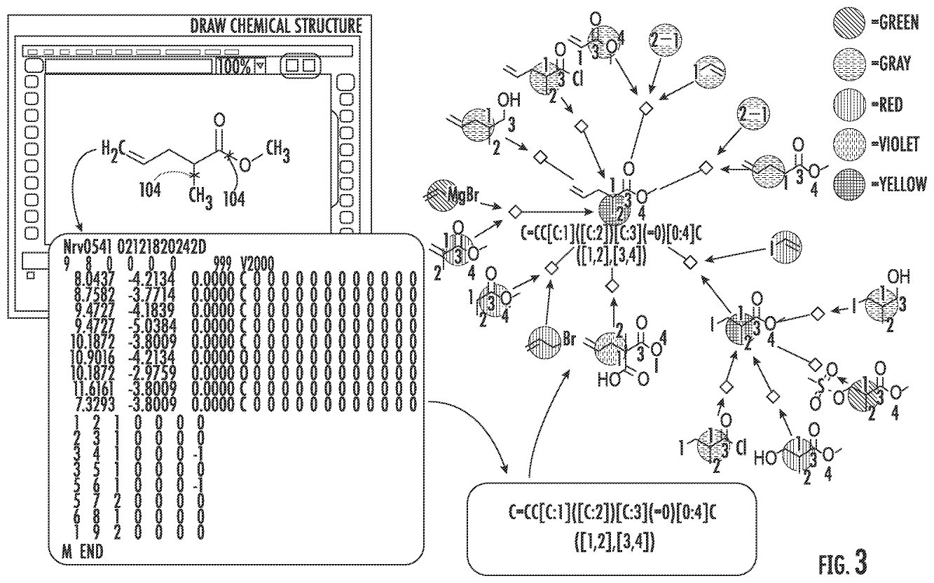

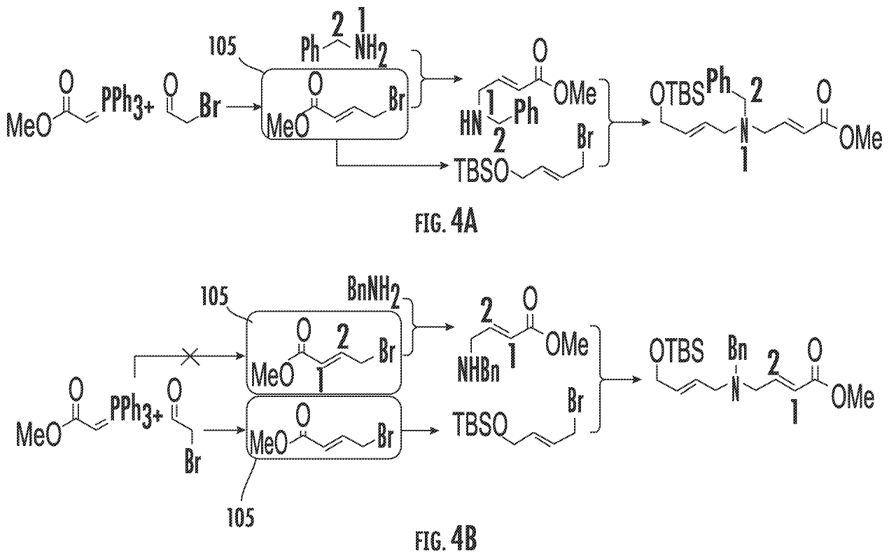

To avoid such problems, the atoms within the motif(s) to be preserved may be numbered. Throughout this disclosure, the term “preserved bonds” is used to represent a bond that is not to be broken. During all generations of retrosynthetic planning, the pairs of atoms corresponding to bonds not to be disconnected are tracked. Importantly, while doing so, search times and memory usage may be minimized by merging the solution space for identical synthons along different putative routes. As seen in the upper left of FIG. 3, the user, using a graphical representation of the target molecule, t, may highlight “preserved bonds” 104. This may be done by selecting the bonds to be preserved with a mouse or other user input device 30. The target molecule, t, with the “preserved bonds” highlighted is first translated into a text file, such as by using Extended Molfile format as shown in the lower left of FIG. 3. In the text file, the “preserved bonds” 104 are uniquely identified. In this illustration, a parameter is given a unique value if this is a “preserved bond” 104. In this example, the value of −1 in the last column reflects that the bond is a “preserved bond”. The file is then translated into a SMILES string, as shown in the lower right of FIG. 3, with atoms belonging to the selected bonds numbered. In certain embodiments, the graphical representation, shown in the upper left is transformed directly to a SMILES string. A list of atom number pairs (“bond list”) denoting the “preserved bonds” 104 is also generated. Specifically, atom labels are stored as SMILES atom index properties and, additionally, a list of pairs indicating “preserved bonds” is created and stored as a “bond set” B(t) . Numbering of atoms in the target molecule t is encoded in its SMILES string (in this example, C=CC[C:1] ([C:2]) [C:3] (═O) [O:4]C) and the accompanying set of atom pairs (denoting “preserved bonds”) is referred to as B(t)={[1,2], [3,4]}) .

When the retrosynthetic search commences, the algorithm inspects to insure that none of the “preserved bonds” 104 (in the SMILES string labelled as [1,2] and [3,4] are disconnected in the synthons. Specifically, the matching reaction templates are applied, and the first generation of synthon sets is created. For each candidate retron-to-synthon(s) transformation, r→s₁, s₂, . . . , s_{N} (where r=t in the first generation), the labels of marked atoms are propagated from the retron to the synthons. A retron is a minimal molecular substructure that enables certain transformations. The algorithm then checks if the set of bonds marked in the target, B(t), is preserved amongst the synthons. Specifically, defining the subset of these bonds in a synthon sᵢ as B(sᵢ), we require that B(r=t)=B(s₁) u . . . u B(s_{N}), where u is a union set operator.

The graph in the upper right of FIG. 3 represents the possible synthesis paths. Each reaction operation (denoted by an open diamond) generates a set of synthons (circles). If the union of bond-sets over the synthons is different than in the target, then such a reaction candidate is removed from further consideration (gray nodes in the graph in the upper right of FIG. 3). In other words, if any “preserved bonds” 104 are disconnected and the bond set changes, such synthetic options are no longer considered.

Only reactions fulfilling this condition are further considered and evaluated. In some embodiments, the most promising options are further expanded into subsequent generations for which the same procedure of atom labelling is applied and to which the same criteria of bond-set conservation are applied. In other embodiments, all such options are further expanded.

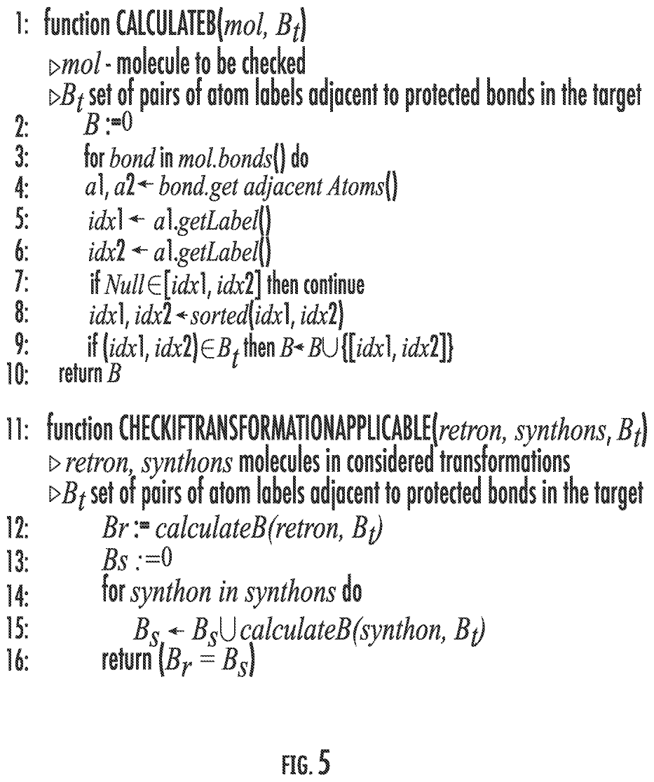

In some embodiments, during consecutive expansions, the searches strive to keep the search space as compact as possible. For instance, it is a relatively frequent scenario that the same synthons are found within different pathways. In this case, they may be stored as one molecule within the search graph. However, if the identical synthons contain different marked bonds, they can possibly have different retrosynthetic histories and are thus stored as separate entities distinguished not by the molecular structure but by the list of “protected” bonds. FIGS. 4A-4B illustrates a search with the same repeating intermediate. When, as shown in FIG. 4A, during the same search, an identical intermediate is encountered several pathways (here, methyl bromocrotonate 105) but does not contain any “preserved bonds”, it is considered as only one node common to different pathways. If, however, as shown in FIG. 4B, if one of bromocrotonates 105 contains “preserved bonds” while the others do not, then synthetic histories for these two molecules may be different and they need to be kept as separate nodes in the search space. In this specific example, a Wittig reaction can be applied to one molecule from the pair but not to the other since it would affect bonds 1-2 marked as “preserved bonds”.

As stated above, for the options that do not destroy the “preserved bonds”, the next-generation nodes are expanded. The remaining synthon nodes can be further expanded (e.g., second-generation expansion on the right) and the search continues until stop conditions are fulfilled. A stop condition is defined as reaching commercially available or previously made chemicals. This are shown as red and green nodes, respectively in the graph in the upper right of FIG. 3. The violet node denotes a new/unknown substance and cannot be a stop point for the search. The graph shown is merely an illustration. An actual graph may have hundreds of potential synthetic options.

In other words, the search continues until all synthons have met user specified criteria. These criteria may include reagents are commercially available, specific reagents are avoided, reaction step(s) described in literature, and so on.

In summary, the algorithm has the following desired characteristics:
(i) it preserves the “preserved bonds” along entire pathways it identifies;
(ii) it can preserve motifs that are disjoint in the target—in such a case, at a given generation, more than one bond-set B(sᵢ) is not empty, meaning that the motifs are split between different synthons;
(iii) it can be implemented to prevent either complete bond disconnections or changes in bond order (the latter, by adding bond-order labels to atom labels).

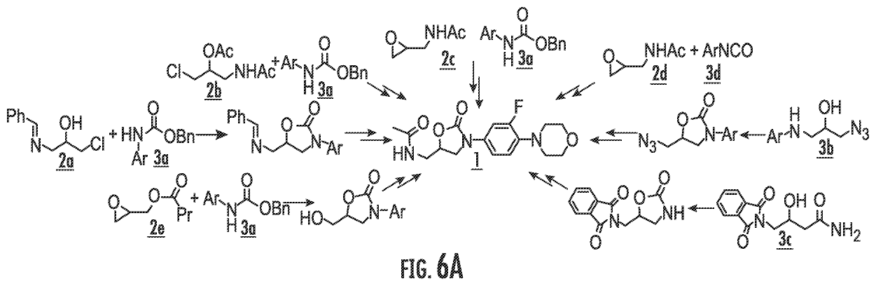

The pseudocode associated with this algorithm in shown in FIG. 5.

The function calculateB (lines 1-10) is used to determine the “preserved bonds” (Bₜ) that are in the molecule mol. For each bond, the pseudocode identifies the two atoms that form that bond. It then gets the label for each atom from the SMILES string. If the bond is not an element within Bₜ, the pseudocode moves onto the next bond in the molecule. If the bond is a “preserved bond”, that bond is added to the set B. At the completion of this routine, B contains the list of bonds within mol that are “preserved bonds”.

At each retrosynthetic step, r→s₁, s₂, . . . , s_{N}, the algorithm applies function checkIfTransformApplicable (lines 11-16) to appropriate retron r, set of synthons s₁, s₂, . . . , s_{N}, and set of “preserved bonds” as defined by the user. The transform is accepted if and only if the following condition is satisfied:

(*)=B(r)=B(s₁) u . . . uB(s_{N}),

where B(m) is a subset of “preserved bonds” in molecule m calculated by function calculateB (lines 1-10). In other words, the pseudocode first determines the set of “preserved bonds” in the retron r and names this set Bᵣ. It then determines the “preserved bonds” in each synthon sᵢ that may be used to create that retron r. The “preserved bonds” from each synthon are incorporated into another set, known as Bₛ. If Bᵣ is the same as Bₛ, this implies that all “preserved bonds” in the retron r are still intact in the synthons sᵢ. Thus, this is considered an acceptable transformation, and a “1” is returned by the function checkIfTransformApplicable.

To explain how the “preserved bonds” are conserved during retrosynthetic search, consider the pathway with the following generations R₀, R₁, . . . , Rₖ, corresponding to sets of synthons available after each step. For the initial generation, R₀={t}, i.e., the search begins from single target molecule. On the other hand Rₖ, the final generation, is composed of synthons that are fulfilling user-defined stop criteria (e.g., all are commercially available).

For retrosynthetic step r→s₁, s₂, . . . , s_{N} leading from Rᵢ₋₁ to Rᵢ we have Rᵢ=Rᵢ₋₁u{s₁, s₂, . . . , s_{N}}\{r} (where \ is a minus operator on sets), namely retron is replaced by the set of synthons. By applying condition (*) as a step constraint, we obtain that U_{s∈R}_{ₖ}B(s)=U_{s∈R}_{_{k−1}}B(s)= . . . =U_{s∈R}_{₀}B(s)=B(t), i.e., the algorithm preserves the “preserved bonds” along entire pathways it identifies.

### EXAMPLE

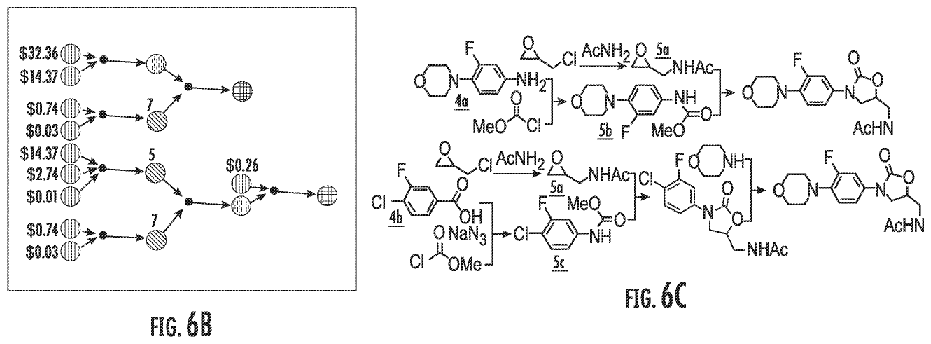

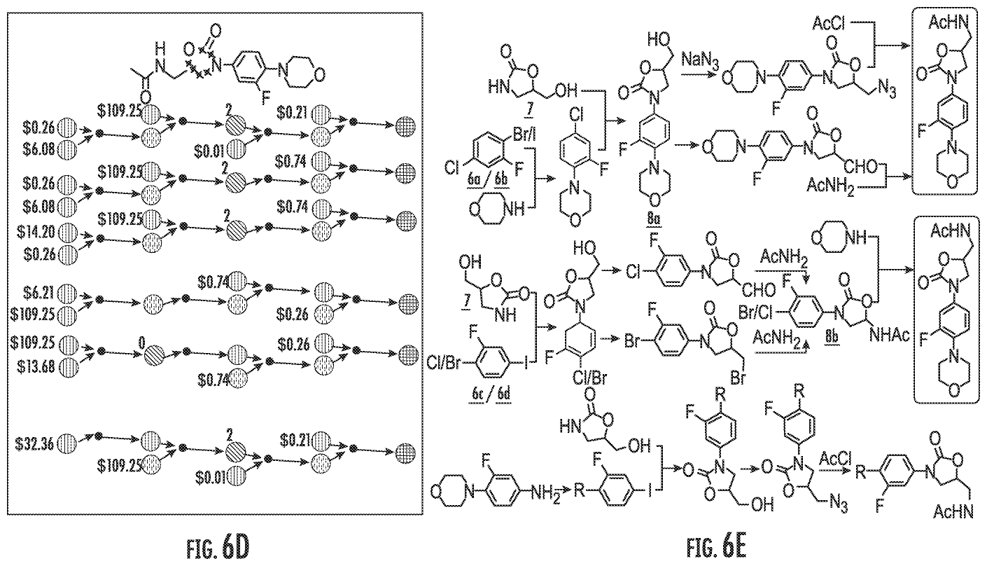

The software application was charged with finding viable new routes leading to the antibiotic linezolid, 1. Referring to FIG. 6a, in the conventional routes, the oxazolidinone ring is formed either via (i) base-induced cyclisation of halohydrin 2a/2b or epoxide 2c/2d/2e with N-aryl carbamate 3a or isocyanate 3d, (ii) cyclisation of 3b, or (iii) Curtius rearrangement of 3c (see FIG. 6a). Without any bond-preservation constraints imposed on the target, the software application proposed similar plans, with the top-scoring pathways (FIGS. 6b, c) constructing oxazolidinone via opening of a known oxirane 5a with carbamate 5b (prepared from appropriate amine 4a) or 5c (prepared via Curtius rearrangement of benzoic acid 4b) and subsequent N-arylation of morpholine (FIG. 6b, c).

In contrast, after specifying the bonds within the oxazolidinone ring as not-to-be-broken (FIG. 6d), the algorithm is forced to avoid the abovementioned key steps, and its three top-scoring solutions (top portion of FIGS. 6d, e) start from commercially available halobenzenes 6a/6b undergoing copper catalyzed amination with morpholine. Subsequent arylation of the commercially available 7 with remaining, less-reactive aryl chloride yields the desired N-aryl oxazolidinone 8a. The four-step sequence is completed by either (i) formation of the azide under Mitsunobu conditions and subsequent one-pot reduction/acylation or (ii) oxidation of the alcohol to the aldehyde followed by the reductive amidation.

Another family of top-scoring computer-generated synthetic plans (middle part of FIG. 6d, e) utilizes an “opposite” reactivity pattern whereby the more reactive aryl iodide 6c/6d is allowed to react with 7. Subsequent (i) conversion to alkyl bromide and reaction with acetamide anion or (ii) oxidation to aldehyde and reductive amidation lead to 8b used in the Buchwald-Hartwig amination of morpholine to complete the synthesis. Finally, the solution shown in the lower portion of FIGS. 6d, e starts from the commercially available fluoroaniline. Conversion via diazonium salt to iodoarene (previously obtained in 85% yield and used for functionalization of cytoxazone) followed by N-arylation of 7, formation of azide, and conversion to acetamide yield the product in four steps.

It is noted that although the catalogue price of 7 (>100 $/g) used as a common intermediate in the applications' plans is rather high, this compound can be prepared in one step from orders-of-magnitude less expensive 3-amino-1,2-propanediol and diethyl carbonate in 60% yield according to literature procedures (see, e.g., K. Danielmeier, E. Steckhan, Tetrahedron: Asymmetry 1995, 6, 1181-1190).

Thus, the present disclosure describes a system, method and software application that allows the user to specify one or more bonds in a target molecule to be preserved. The system, method and software application then produce the various synthesis paths that result in the target molecule that do not break the “preserved bonds”.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A method of performing retrosynthesis on a target compound wherein certain bonds are preserved, comprising:
identifying bonds in the target compound that are to be preserved;
setting the target compound to a retron;
performing a first retrosynthesis search on the retron to find a set of synthons;
determining if the bonds are preserved across the set of synthons;
discarding the set of synthons if the bonds are not preserved; and
if the bonds are preserved, setting the set of synthons to the retron and repeating the performing, determining, and discarding steps.

2. The method of claim 1, wherein the certain bonds are preserved so as to avoid a particular synthon.

3. The method of claim 2, wherein the particular synthon is a patented compound.

4. The method of claim 1, wherein the setting, performing, determining and discarding steps are repeated until all synthons meet user specified criteria.

5. The method of claim 4, wherein the user specified criteria comprises all synthons are commercially available.

6. A software program, disposed on a non-transitory storage media, the software program comprising instructions, which when executed by a processing unit perform retrosynthesis on a target compound wherein certain bonds are preserved, by:
identifying bonds in the target compound that are to be preserved;
setting the target compound to a retron;
performing a first retrosynthesis search on the retron to find a set of synthons;
determining if the bonds are preserved across the set of synthons;
discarding the set of synthons if the bonds are not preserved; and
if the bonds are preserved, setting the set of synthons to the retron and repeating the performing, determining, and discarding steps.

7. The software program of claim 6, wherein the setting, performing, determining and discarding steps are repeated until all synthons meet user specified criteria.

8. The software program of claim 7, wherein the user specified criteria comprises all synthons are commercially available.

9. The software program of claim 6, wherein the bonds of each synthon in the set of synthons are identified and wherein determining if the bonds are preserved across the set of synthons comprises comparing the bonds identified in each synthon in the set of synthons to the bonds that are to be preserved.

10. The software program of claim 6, wherein the bonds that are to be preserved are identified based on labels assigned in a SMILES string.
